(19) United States
(12) Patent Application Publication
(10) Pub. No.: US 2004/0092519 A1
(43) Pub. Date: May 13, 2004

(54) New treatment for hot flashes

(76) Inventor: Fred Hassan, Kenilworth, NJ (US)

Correspondence Address: PHARMACIA & UPJOHN, 301 HENRIETTA ST, 0228-32-LAW, KALAMAZOO, MI, 49007, US

(21) Appl. No.: 10/633,762

(22) Filed: Aug. 4, 2003

Related U.S. Application Data
(60) Provisional application No. 60/403,549, filed on Aug. 14, 2002.

(51) Int. Cl. A61K 31/537

(52) U.S. Cl. 514/238.2

(57) ABSTRACT

This patent application describes a method for treating or preventing hot flashes in a patient suffering therefrom comprising administering a therapeutically effective dose of a compound selected from reboxetine, S,S-reboxetine, pharmaceutically acceptable salts thereof, derivatives thereof, or prodrugs thereof to the patient.

### CROSS REFERENCE

This application claims the benefit of the following provisional application: U.S. Serial No. 60/403,549, filed Aug. 14, 2003 under 35 USC 119(e)(i), which is incorporated herein by reference in its entirety.

### FIELD OF THE INVENTION

This invention describes a new treatment for hot flashes. The treatment involves the administration of the drug reboxetine, pharmaceutically acceptable salts thereof, derivatives thereof, or prodrugs thereof to a patient in need thereof

### BACKGROUND OF THE INVENTION

Hot flashes are a common complaint. The patient experiences a sudden onset of heat, which generally starts in the face and then can progress to the neck, chest and the rest of the body. Often the attacks are accompanied by a red flush of the skin and/or profuse sweating. These attacks, which can occur several times a day, can be exceedingly uncomfortable to the person experiencing them.

Although the exact cause of hot flashes is not known, they are often attributed to an imbalance of the patient's hormone system. A large group of patients, who experience hot flashes, are menopausal women. To date, this group of patients has often received estrogens or hormone replacement therapy to alleviate or prevent menopause symptoms, including hot flashes (E. Daly et al., Br. Med. J. 1993; 307:836-840). However, some women are reluctant to agree to a hormone therapy. A range of “natural” therapies on a herbal basis including black cohosh, phytoestrogens, flax seed, red clover, vitamin E (D. L. Barton et al., J. Clin. Oncol. 1998, 16: 495-500), ginseng and evening primrose oil have been advocated as possible medications (University of Wisconsin Medical School, online courses, “Alternatives for Menopausal Symptoms: A Review of the Evidence”; www.cme.wisc.edu/online/menopause). However, not all of these therapies are effective (K. I. Pritchard, The Oncologist, 2001, 6(4), 353-362).

Other medications, which have been suggested, are selective serotonin reuptake inhibitors (SSRIs) such as fluoxetine hydrochloride (Prozac; C. Loprinzi; www.medicine-news.com/articles/pharma/misc/hotflashes.html) and paroxetine hydrochloride (Paxil; V. Stearns et al., Ann. Oncol., 2000, 11:17-22) as well as venlafaxine hydrochloride (Effexor; C. L. Loprinzi et al., J. Clin. Oncol., 1998, 16: 2377-2381), which is a serotonin and norepinephrine reuptake inhibitor.

Low doses of megestrol acetate have also been shown to reduce the frequency of hot flashes in both men and women (Loprinzi et al., N. Engl. J. Med. 1994, 331:347-351). Chronic adrenal insufficiency and weight gain can be side effects. Transdermal clonidine has also been employed to reduce the frequency and severity of hot flashes (R. M. Goldberg et al., J. Clin. Onc. 1994, 12:155-158); R. M. Goldberg et al., J. Clin. Oncol. 1994, 12:155-158; L. R. Laufer, Obstet. Gynecol. 1982, 60:583-586). However, side effects such as drowsiness, fatigue, and symptoms of low blood pressure in some patients were observed.

Both men and women can suffer from hot flashes as a side effect of cancer therapy. Certain drugs such as Tamoxifen (Nolvadex), which is used to treat breast cancer, as well as Lupron (Leuprolide) and Zoladex (Goserelin), which are employed in the therapy of prostate cancer, can lead to heat sensations. Bilateral orchiectomy for prostate cancer or testicular cancer also affects the hormone system so that patients can subsequently suffer from hot flashes. Especially in the case of cancer patients, hormone replacement therapy is often not advised, because there is a concern that cancer regrowth can be stimulated.

In view of the disadvantages of the prior art, there remains a need for further medications, which can reduce the number and/or severity of hot flashes. It has now been found that reboxetine is effective in treating these attacks.

### SUMMARY OF THE INVENTION

The present invention provides a method of treating and/or preventing hot flashes in a patient suffering therefrom comprising administering a therapeutically effective dose of a compound selected from reboxetine, pharmaceutically acceptable salts thereof, derivatives thereof, or prodrugs thereof to the patient.

In a further embodiment the use of a compound selected from reboxetine, pharmaceutically acceptable salts thereof, derivatives thereof, or prodrugs thereof for the manufacture of a medicament to treat and/or prevent hot flashes is disclosed.

The present invention also refers to a method of treating and/or preventing a symptom of hormonal variation in a patient suffering therefrom comprising administering a therapeutically effective dose of a compound selected from reboxetine, an enantiomer or diasteromer, a pharmaceutically acceptable salt thereof, a derivative thereof, or a prodrug thereof to the patient.

### DETAILED DESCRIPTION OF THE INVENTION

Reboxetine is the generic name of the pharmaceutical substance with the chemical name of 2-[α-(2-ethoxy)phenoxybenzyl]morpholine, and its pharmaceutically acceptable salts. Reboxetine is also known under the trade names of VESTRA, EDRONAX, PROLIFT, INTEGREX, and NOREBOX. Besides the racemic mixture of R,R- and S,S-enantiomers, preferably the pure S,S-enantiomer can be employed in the present invention.

Reboxetine acts as an antidepressant. Antidepressants are frequently grouped into categories or “generations”. The first generation of antidepressants were usually tricyclic antidepressants such as maprotiline that affected various neurotransmitter systems and are associated with many undesirable side effects. The second generation of antidepressants, such as mianserine, mirtrazapine and trazodone are largely devoid of anticholinergic action and their adrenolytic and antihistaminic effects are weaker. These are contrasted with the third generation of antidepressants (e.g. SSRI, ipsapirone, viloxazine, reboxetine, bupropione) that mediate only one of the three main neurotransmitter systems for depression (5-HT, noradrenaline, dopamine) and they do not affect muscarine, histamine and adrenergic cerebral systems. J. Svestka. “Antidepressives of the 3rd, 4th and 5th generation”, Cesk-Psychiatr. 1994 Febuary; 90(1):3-19(Czech).

Reboxetine, however, does not act like most antidepressants. Unlike tricyclic antidepressants and even selective serotonin reuptake inhibitors (SSRIs), reboxetine is ineffective in the 8-OH-DPAT hypothermia test, indicating that reboxetine is not a selective serotonin reuptake inhibitor but rather that it is selective for the noradrenergic system. Thus, reboxetine is not an SSRI, rather it is considered a novel, selective, noradrenaline-reuptake inhibitor (NARI). B. E. Leonard, “Noradrenaline in basic models of depression”. European-Neuropsychopharmacol. 1997 April; 7 Suppl 1: S11-6; discussion S71-3. Unlike most drugs, reboxetine is a highly selective norepinephrine uptake inhibitor, with only marginal serotonin and no dopamine uptake inhibitory activity. The compound displays only weak or no anti-cholinergic activity in different animal models and is devoid of monoamine oxidase (MAO) inhibitory activity.

Reboxetine is highly potent and fast acting. Our investigations indicate that reboxetine has potent antireserpine activity and combines the inhibitory properties of classical tricyclic antidepressants on the reuptake of noradrenaline with an ability to desensitize J-adrenergic receptor finction without showing any appreciable interaction with muscarinic cholinergic and I-adrenerigic receptors. Moreover, reboxetine shows less vagolytic activity than other tricyclic antidepressants.

The inventors have discovered that, because of its unique properties, reboxetine is particularly useful for treating or preventing hot flashes. Furthermore, the inventors have discovered that reboxetine can be used to treat or prevent symptoms of hormone variation in a patient.

In the present invention reboxetine can be employed in its free base form. Furthermore, reboxetine methanesulfonate (also called reboxetine mesylate) or any other pharmaceutically acceptable salt that does not significantly affect the pharmaceutical activity of the substance can be used such as the succinate or fumarate salt thereof. The use of pharmaceutically acceptable derivatives as well as of prodrugs of reboxetine is also possible. The expression “prodrug” denotes a derivative of a known direct acting drug, which derivative has enhanced delivery characteristics and therapeutic value as compared to the drug, and is transformed into the active drug by an enzymatic process, for example by hydrolysis in blood, or a chemical process [see H. Bundgaard, “Design of Prodrugs: Bioreversible-Derivatives for Various Functional Groups and Chemical Entities”, in Design of Prodrugs (H. Bundgaard, ed.), Elsevier, N.Y. (1985)].

Reboxetine and its various derivatives and a method of synthesis therefore are described in U.S. Pat. No. 4,229,449 (Melloni et. al.), which is incorporated herein by reference. Methods of preparing reboxetine are also described in U.S. Pat. No. 5,068,433 (Melloni et. al.) and in U.S. Pat. No. 5,391,735 (Melloni et. al.), both of which are incorporated by reference.

Reboxetine is useful in treating or preventing hot flashes by reducing the number and/or severity of the attacks. The hot flashes treated according to the invention can be due to a number of causes. Reboxetine can be employed to treat or prevent hot flashes, which occur as a symptom of the postmenopause phase, but it is also effective if the hot flashes have other causes. In particular, various medical therapies can imbalance the hormone system of both female and male patients resulting in attacks of hot flashes.

Female patients having a low level of estrogen are prone to suffer from hot flashes. This deficiency can be due to radiation therapy, which can prematurely induce the menopause, or can be caused by specific medications such as anti-estrogen treatment or certain drugs (e.g. Tamoxifen (Nolvadex)).

Androgen deprivation can be a cause of hot flashes in men. Again the imbalance of the hormone system can be drug-induced (e.g. Lupron (Leuprolide) and Zoladex (Goserelin)) or be radiation-induced. Surgery such as bilateral orchiectomy for prostate cancer or testicular cancer is a further possible cause.

Reboxetine can be administered to the patient in the form of a pharmaceutical composition. Pharmaceutical compositions and methods of administration, which are useful in the present invention, are described, for example, in U.S. Pat. No. 4,229,449 at col. 18, lines 33-66. This reference is specifically incorporated herein by reference. Pharmaceutically acceptable carriers and excipients as well as other adjuvants are known in the art and can be selected based on the desired route of administration.

Reboxetine can be administered in a dose range of active ingredient from about 1 to over 20 mg/kg. It is more commonly provided in dosages of from 1 to 20 mg per patient per day. The compound may be administered by any suitable method including a convenient oral dosage form. A preferred method is oral dosing twice a day. The preferred dose range is 4 to 10 mg per patient per day and the most preferred dose is 6 to 8 mg or 8 to 10 mg per patient daily, depending upon the patient, delivered twice a day (b.i.d.). It can also be given at dosages of 2, 4, 6, 8, 10 or 12 mg per patient per day or fractions thereof. For example, suitable administrations could be 4 mg in the morning and 2 or 4 mg in the evening or 6 mg in the morning and 4 mg in the evening. In some patients the ideal dosing would be 3-5 mg in the morning and 3-5 mg in the evening. A skilled practitioner would be expected to determine the precise level of dosing. The ideal dosing would be routinely determined by an evaluation of clinical trials and the needs of the patient.

Reboxetine is effective in treating hot flashes. It is especially useful for treating patients who are suffering from,or who have suffered from cancer and consequently should not receive hormone replacement therapy. The present invention now provides a novel and safe method of treating these undesirable attacks.

1. A method for treating or preventing hot flashes in a patient in need thereof comprising administering a therapeutically effective dose of a compound selected from reboxetine or S,S-reboxetine, a pharmaceutically acceptable salt thereof, a derivative thereof, or a prodrug thereof to the patient.

2. A method of claim 1, wherein the patient is female.

3. A method according to claim 2, wherein the hot flashes are menopause or postmenopause symptons.

4. A method according to claim 2, wherein the hot flashes are due to medical treatment.

5. A method according to claim 2, wherein the hot flashes are caused by radiation therapy.

6. A method according to claim 2, wherein the hot flashes are drug-induced.

7. A method according to claim 2, wherein the patient is receiving anti-estrogen therapy.

8. A method according to claim 2, wherein the patient is suffering from or has suffered from cancer.

9. A method according to claim 5, wherein the cancer is breast cancer.

10. A method according to claim 1, wherein the patient is male.

11. A method according to claim 10, wherein the hot flashes are caused by radiation therapy.

12. A method according to claim 10, wherein the hot flashes are drug-induced.

13. A method according to claim 10, wherein the patient has androgen deprivation.

14. A method according to claim 10, wherein the patient is suffering from or has suffered from cancer.

15. A method according to claim 14, wherein the cancer is prostate cancer or testicular cancer.

16. The method according to claim 1, wherein the reboxetine dose range is 4 to 10 mg per patient per day.

17. The method according to claim 1, wherein the reboxetine dose range is 6 to 8 mg per patient per day.

18. The method according to claim 1, wherein the compound is administered in the form of a pharmaceutical composition additionally comprising a pharmaceutically acceptable carrier or excipient.

19. The use of a compound selected from reboxetine, a pharmaceutically acceptable salts thereof, a derivative thereof, or a prodrug thereof for the manufacture of a medicament to treat or prevent hot flashes.

20. The use according to claim 19, wherein the reboxetine dose range is 4 to 10 mg per patient per day.

21. The use according to claim 19, wherein the reboxetine dose range is 6 to 8 mg per patient per day.

22. A method for treating or preventing symptoms of hormonal variation in a patient suffering therefrom comprising administering a therapeutically effective dose of a compound selected from reboxetine, a pharmaceutically acceptable salt thereof, a derivative thereof, or a prodrug thereof to the patient.
